(19) United States
(12) Patent Application Publication
(10) Pub. No.: US 2024/0325273 A1
(43) Pub. Date: Oct. 3, 2024

(54) COMPOSITION COMPRISING A SPECIFIC COMBINATION OF SURFACTANTS AND A CATIONIC POLYMER

(71) Applicant: L'Oreal, Paris (FR)

(72) Inventors: Anne-Laure Fameau, Saint Ouen (FR); Saber Maloug, Saint Ouen (FR); Marjorie Rochard, Saint Ouen (FR); Christian Tu, Saint Ouen (FR)

(73) Assignee: L'Oreal, Paris (FR)

(21) Appl. No.: 18/580,525

(22) PCT Filed: Jul. 19, 2022
(86) PCT No.: PCT/EP2022/070246

§ 371 Date: Jan. 18, 2024

(30) Foreign Application Priority Data
Jul. 19, 2021 (FR) FR2107754

(51) Int. Cl. A61K 8/60; A61K 8/37; A61K 8/41; A61K 8/46; A61K 8/73; A61Q 5/02; A61Q 5/12

(52) U.S. Cl. CPC A61K 8/604; A61K 8/375; A61K 8/416; A61K 8/463; A61K 8/737; A61Q 5/02; A61Q 5/12; A61K 2800/5426; A61K 2800/596

(57) ABSTRACT

Composition comprising a specific combination of surfactants and a cationic polymer The present invention relates to an aqueous composition intended in particular for washing and/or conditioning keratin fibres, notably human keratin fibres such as the hair, and which comprises at least one anionic surfactant, the combination of at least two nonionic surfactants, different from one another, in a specific weight ratio, at least one amphoteric surfactant and at least one cationic polymer. The invention also relates to a method for the cosmetic treatment of keratin fibres, in particular human keratin fibres such as the hair, using said composition. The invention further relates to the use of said composition for washing and/or conditioning keratin fibres, in particular human keratin fibres such as the hair.

The present invention relates to a composition intended in particular for washing and/or conditioning keratin fibres, notably human keratin fibres such as the hair, and which comprises at least one anionic surfactant, the combination of at least two nonionic surfactants, different from one another, in a specific weight ratio, at least one amphoteric surfactant and at least one cationic polymer.

The invention also relates to a method for the cosmetic treatment of keratin fibres, in particular human keratin fibres such as the hair, using said composition.

The invention further relates to the use of said composition for washing and/or conditioning keratin fibres, in particular human keratin fibres such as the hair.

Keratin fibres, notably the hair, have a tendency to lose some of their qualities due to the action of various factors such as for example natural regreasing, sweat, the removal of squamae, pollution or humidity. The visual appearance and the feel of the fibres may be degraded thereby. Regreasing, for example, makes the fibers lank, which then have a tendency to clump together. The hair may then be more difficult to style, and have an unpleasant greasy sheen and/or waxy feel.

It is common practice to use detergent cosmetic compositions such as shampoos based essentially on surfactants, for washing keratin fibres, such as the hair. These compositions are generally applied to the keratin fibres, which are preferably wet, and the foam generated by massaging or rubbing with the hands or a washing mitt makes it possible, after rinsing with water, to remove the diverse types of soiling initially present on the hair.

Consumers are increasingly in search of natural hair and body care products, the raw materials of which originate from natural sources such as plants. The natural compositions currently marketed certainly have good washing power but the cosmetic and conditioning properties which are attached thereto however remain to be improved.

Furthermore, the supply of opacified, notably nacreous, natural detergent compositions is still too low. Indeed, these natural compositions do not generally have a good ability to suspend opacifying agents and are not stable enough over time, or with respect to temperature. They notably have a tendency to phase separate rapidly, which makes their application difficult and inhomogeneous and gives the consumer an unpleasant experience.

In order to improve the usage qualities (homogeneity, ease of application), it is necessary for the composition to have a certain viscosity or rheology. On an industrial scale, the viscosity is generally adjusted by the addition of salt (NaCl for example). However, this adjustment of the viscosity of the natural compositions currently on sale is particularly difficult due notably to their low reactivity to salt.

Thus, there is a real need to provide a composition, preferably a natural composition, intended in particular for washing and/or conditioning keratin fibres, which has a pleasant and thick texture which remains stable over time. The composition must also have good foaming properties, notably in terms of the start of foaming and the foam abundance and density, and also good detergent power.

The composition must also impart satisfactory cosmetic and conditioning properties to the keratin fibres, notably in terms of manageability, a smooth feel or smooth appearance, softness, suppleness, sheen, disentangling, strand separation and lightness.

More particularly, the composition must impart smoothness and suppleness to the keratin fibres.

It is now been discovered that a composition comprising a specific combination of surfactants in the presence of at least one cationic polymer when it possible to achieve the objectives disclosed above, and notably to provide a composition that is stable over time, combining foaming and detergent power with improved cosmetic and conditioning properties (in particular, smoothness and suppleness to the keratin fibres).

One subject of the present invention is therefore a composition comprising:
(i) one or more anionic surfactants,
(ii) one or more nonionic surfactants of alkyl (poly)glycoside type,
(iii) one or more polyglycerol esters,
(iv) one or more amphoteric or zwitterionic surfactants, and
(v) one or more cationic polymers;
wherein the total content of water ranges from 60% to 99% by weight, relative to the total weight of the composition, and
the weight ratio between the total content of nonionic surfactants of alkyl (poly)glycoside type (ii) and the total content of polyglycerol esters (iii) is less than or equal to 1.

The specific combination of the compounds of the invention makes it possible to obtain a composition that has good foaming properties, and also good washing power. Specifically, the composition of the invention gives a firm, creamy and abundant foam.

It rinses out rapidly without leaving unpleasant residues on the fibres and gives them a natural, clean feel after rinsing. Fibres treated with the composition of the invention also have good cosmetic properties, notably in terms of softness, manageability, suppleness, smooth feel and smooth appearance. They also have good strand separation, are light and are easier to disentangle.

In particular, it has been discovered that the weight ratio of the total content of nonionic surfactants of alkyl (poly)glycoside type (ii) to the total content of polyglycerol esters (iii) less than or equal to 1, makes it possible to obtain improved cosmetic and conditioning properties, and more particularly to obtain smoother and more supple hair.

Furthermore, the composition of the invention has a thickened texture enabling an easy and homogeneous distribution over all of the keratin fibres, and notably the hair, while avoiding running into the eyes for example. Despite its thick texture, the composition of the invention is also particularly stable over time, and also with respect to temperature.

By implementing the invention, it is thus possible to obtain foaming and thickened natural compositions or compositions of natural origin without having to add large amounts of thickeners other than salt (for example NaCl), which might harm the stability of said composition and/or its foaming properties.

Another subject of the present invention is a method for cosmetic treatment, in particular for washing and/or conditioning, of keratin fibres, notably human keratin fibres such as the hair, comprising the application to said keratin fibres of a composition as defined previously; said application optionally being followed by rinsing after an optional leave-on time.

The present invention also relates to the use of a composition as defined previously for washing and/or conditioning keratin fibres, in particular human keratin fibres such as the hair.

Other subjects, features, aspects and advantages of the invention will become even more clearly apparent on reading the description and the example which follows.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

According to the present invention, the term “stable over time” is intended to mean a composition which, after one month, preferably after two months, of storage at a temperature ranging from 4° C. to 45° C., does not exhibit any macroscopic change, notably in odour or in viscosity, or any variation in pH or any phase separation, and also no variation in microscopic appearance.

The composition of the invention preferably comprises ingredients of natural origin.

The composition according to the invention has a viscosity, measured at 25° C. and 1 atm, preferably ranging from 20 to 70 seconds, measured with a Ford cup 10. The viscosity is measured with a Ford cup. The Ford cup standard corresponds to a viscosity between 3.7 and 6.4 Pa·s for a velocity gradient of 10 s⁻¹.

The composition of the invention is preferably opaque.

For the purposes of the present invention, the term “opaque composition” is intended to mean a composition through which it is impossible to see clearly with the naked eye. In particular, the composition according to the invention may have a turbidity value advantageously greater than or equal to 200 NTU units, preferably greater than or equal to 300 NTU units, and more preferentially greater than or equal to 350 NTU units. The turbidity may be measured according to the NTU method, using a 2100P model turbidimeter from the company Hach Co., at room temperature and pressure (25° C. and 1 atm.).

The opacity of the composition may also be characterized by measuring its transmittance, which is measured using a Cary 100 model spectrophotometer from the company Varian, at room temperature and pressure (25° C., 1 atm.), at a wavelength of 700 nm. The transmittance of the compositions according to the invention is preferably less than or equal to 80%.

### (i) The Anionic Surfactants

The composition according to the present invention comprises one or more anionic surfactants.

The term “anionic surfactant” means a surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactant(s) (i) may be chosen from sulfate, sulfonate and/or carboxylic (or carboxylate) surfactants. Needless to say, a mixture of these surfactants may be used.

It is understood in the present description that:
the carboxylate-type anionic surfactants comprise at least one carboxylic or carboxylate function (—COOH or —COO⁻) and may optionally also comprise one or more sulfate and/or sulfonate functions;
the sulfonate-type anionic surfactants comprise at least one sulfonate function (—SO₃H or —SO₃⁻) and may optionally also comprise one or more sulfate functions, but do not comprise any carboxylate functions; and
the sulfate-type anionic surfactants comprise at least one sulfate function but do not comprise any carboxylate or sulfonate functions.

The carboxylate-type anionic surfactants that may be used in the composition of the invention thus include at least one carboxylic or carboxylate function (—COOH or —COO⁻).

They may be chosen from the following compounds: acyl glycinates, acyl lactylates, acyl sarcosinates, acyl glutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;

the alkyl and/or acyl groups of these compounds including from 6 to 30 carbon atoms, preferably from 8 to 26, and more preferentially from 10 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Use may also be made of C₆-C₃₀ alkyl monoesters of polyglycoside-polycarboxylic acids such as C₆-C₃₀ alkyl polyglycoside-citrates, C₆-C₃₀ alkyl polyglycoside-tartrates and C₆-C₃₀ alkyl polyglycoside-sulfosuccinates, and salts thereof.

Preferentially, the carboxylate anionic surfactants are chosen, alone or as a mixture, from:
acyl glutamates, notably C₆-C₃₀ or even C₈-C₂₆ acyl glutamates, such as stearoyl glutamates, and in particular disodium stearoyl glutamate;
acyl sarcosinates, notably C₆-C₃₀ or even C₈-C₂₆ acyl sarcosinates, such as palmitoyl sarcosinates, and in particular sodium palmitoyl sarcosinate;
acyl lactylates, notably C₆-C₃₀ or even C₈-C₂₆ acyl lactylates, such as behenoyl lactylates, and in particular sodium behenoyl lactylate;
C₆-C₃₀ and notably C₈-C₂₆ acyl glycinates;
(C₆-C₃₀)alkyl ether carboxylates, and notably (C₈-C₂₆)alkyl ether carboxylates;
polyoxyalkylenated (C₆-C₃₀)alkyl(amido) ether carboxylic acids, in particular those including from 2 to 50 ethylene oxide groups;

in particular in the form of alkali metal or alkaline-earth metal, ammonium or amino alcohol salts.

Among the above carboxylate-type surfactants, mention may be made most particularly of surfactants of fatty acid type, notably of C₆-C₃₀. These surfactants are preferably chosen from the compounds of formula (a) below:

R—C(O)—OX (a)

with
X denoting a hydrogen atom, an ammonium ion, an ion resulting from an alkali metal or alkaline-earth metal or an ion resulting from an organic amine, preferably a hydrogen atom, and
R denoting a linear or branched, saturated or unsaturated alkyl group of 5 to 29 carbon atoms.

Preferably, R denotes a linear or branched, saturated or unsaturated alkyl group of 7 to 23 carbon atoms, preferably of 11 to 21 carbon atoms.

Among the above carboxylate-type surfactants, mention may be made most particularly of surfactants of sarcosinate type, notably chosen from the (C₆-C₃₀)acyl sarcosinates of formula (I) below:

R—C(O)—N(CH₃)—CH₂—C(O)—OX (I)

with
X denoting a hydrogen atom, an ammonium ion, an ion resulting from an alkali metal or alkaline-earth metal or an ion resulting from an organic amine, preferably a hydrogen atom, and
R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that can be used in the present composition, mention may be made of palmitoyl sarcosinates, stearoyl sarcosinates, myristoyl sarcosinates, lauroyl sarcosinates and cocoyl sarcosinates, in acid form or in salified form.

Among the above carboxylate-type surfactants, mention may also be made of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (II):

R1-(OC₂H₄)ₙ—OCH₂COOA (II)

in which:
R1 represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;
preferably, R1 is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (II), in particular mixtures of compounds bearing different groups R1.

The polyoxyalkylenated alkyl(amido) ether carboxylic acids that are particularly preferred are those of formula (II) in which:
R1 denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (II) in which R1 denotes a C₁₂ alkyl radical, A denotes a hydrogen or sodium atom and n ranges from 2 to 10.

The sulfonate-type anionic surfactants that may be used in the composition of the invention include at least one sulfonate function (—SO₃H or —SO₃⁻).

They may be chosen from the following compounds: alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, N-acyl taurates, acyl isethionates; alkyl sulfolaurates; and also the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyl taurates;
(C₆-C₂₄)acyl isethionates, preferably (C₁₂-C₁₈)acyl isethionates;
   in particular in the form of alkali metal or alkaline-earth metal, ammonium or amino alcohol salts.

Preferably, the surfactant(s) (i) are chosen from sulfate-type anionic surfactants and mixtures thereof, and more preferentially from non-oxyalkylenated sulfate-type anionic surfactants, also referred to as sulfated non-oxyalkylenated anionic surfactants, and mixtures thereof.

For the purposes of the present invention, the term “sulfate-type anionic surfactant” means an anionic surfactant including one or more sulfate functions (—OSO₃H or —OSO₃⁻).

For the purposes of the present invention, the term “non-oxyalkylenated surfactant” means a surfactant that does not comprise any alkylene oxide unit, in particular any ethylene oxide (OCH₂CH₂) unit, propylene oxide unit or butylene oxide unit.

The anionic surfactant(s) (i) may advantageously be chosen from alkyl sulfates, alkyl ether sulfates, alkylamido sulfates, alkylamido ether sulfates, alkylaryl polyether sulfates, monoglyceride sulfates; and also salts thereof and mixtures thereof; the alkyl groups of these compounds notably including from 8 to 30 carbon atoms, preferably from 8 to 26, and more preferentially from 10 to 22 carbon atoms; the aryl group preferably denoting a phenyl orif benzyl group.

Preferably, the anionic surfactant(s) (i) are chosen from:
alkyl sulfates, notably C₈ to C₂₆, and preferably C₁₀ to C₂₂, alkyl sulfates;
alkyl ether sulfates, notably C₈ to C₂₆ and preferably C₁₀ to C₂₂ alkyl ether sulfates, preferably comprising from 1 to 10 ethylene oxide units;

in particular in the form of alkali metal, alkaline-earth metal, ammonium or amino alcohol salts; and

mixtures thereof.

More preferentially, the anionic surfactant(s) (i) are chosen from alkyl sulfates, notably C₈ to C₂₆, and preferably C₁₀ to C₂₂, alkyl sulfates in particular in the form of alkali metal, alkaline-earth metal, ammonium or amino alcohol salts, and mixtures thereof.

When the anionic surfactant(s) (i) are in salt form, said salt may be chosen from alkali metal salts, such as the sodium or potassium salt, ammonium salts, amine salts and in particular amino alcohol salts, and alkaline-earth metal salts, such as the magnesium salt, and mixtures thereof.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferably, the anionic surfactant(s) (i) are chosen from sodium, triethanolamine, magnesium or ammonium (C₁₀-C₂₂)alkyl sulfates, sodium, ammonium or magnesium (C₁₀-C₂₂)alkyl ether sulfates, and mixtures thereof.

Even better still, the anionic surfactant(s) (i) are chosen from sodium, triethanolamine, ammonium or magnesium (C₁₀-C₂₂)alkyl sulfates, such as the compound sold under the name Texapon Z95P by the company BASF under the INCI name Sodium lauryl sulfate.

Advantageously, the anionic surfactant(s) (i) are chosen from sodium or ammonium lauryl sulfate, sodium or ammonium cetearyl sulfate, sodium or ammonium coco sulfate, and mixtures thereof. Preferably, the anionic surfactants (i) is sodium lauryl sulfate.

The total content of the anionic surfactant(s) (i) present in the composition of the invention preferably ranges from 1% to 30% by weight, and more preferentially from 2% to 20% by weight, and even better still from 5% to 10% by weight, relative to the total weight of the composition.

In one particular embodiment of the invention, the composition comprises one or more, preferably non-oxyalkylenated, sulfate-type anionic surfactants. According to this embodiment, the total content of the preferably non-oxyalkylenated, sulfate-type anionic surfactant(s) preferably ranges from 1% to 30% by weight, and more preferentially from 2% to 20% by weight, and even better still from 5% to 10% by weight, relative to the total weight of the composition.

### (ii) Nonionic Surfactants of Alkyl (Poly)Glycoside Type

The composition according to the present invention further comprises one or more nonionic surfactants of alkyl (poly)glycoside (or APG) type, also referred to as alkyl (poly)glycoside nonionic surfactants.

The term “alkyl (poly)glycoside” denotes an alkyl polyglycoside or an alkyl monoglycoside, also referred to in the present patent application as an alkyl glycoside, which may be alkoxylated with one or more alkylene oxide groups, preferentially C₂-C₄alkylene oxide groups.

The nonionic surfactant(s) of alkyl (poly)glycoside type (ii) are preferably chosen from the compounds of formula (III) below and mixtures thereof:

R₁O—(R₂O)ₜ(G)ᵥ (III)

in which formula (I):
R₁ represents a linear or branched, saturated or unsaturated alkyl group including from 6 to 24 carbon atoms, or an alkylphenyl group in which the linear or branched alkyl group includes from 6 to 24 carbon atoms,
R₂ represents an alkylene group including from about 2 to 4 carbon atoms,
G represents a saccharide unit including 5 or 6 carbon atoms,
t denotes a value ranging from 0 to 10 and preferably 0 to 4, and
v denotes a value ranging from 1 to 15.

Preferably, the nonionic surfactant(s) of alkyl (poly)glycoside type are chosen, alone or as a mixture, from the compounds of formula (III) in which:
R₁ denotes a linear or branched, saturated or unsaturated alkyl group including from 8 to 18 carbon atoms,
G denotes glucose, fructose or galactose, preferably glucose,
t denotes a value ranging from 0 to 3, and is preferably equal to 0, and
R₂ and v are as defined previously.

The degree of polymerization of the alkyl (poly)glycoside nonionic surfactant(s) as represented, for example, by the subscript v in formula (III) above ranges on average from 1 to 15 and preferably from 1 to 4. This degree of polymerization more particularly ranges from 1 to 2 and even better still from 1.1 to 1.5, on average.

The glycoside bonds between the saccharide units are 1,6- or 1,4-bonds; preferably 1,4-bonds.

The alkyl (poly)glycoside nonionic surfactants that may be used in the present invention are preferably alkyl (poly)glycosides notably represented by the products sold by the company Cognis under the names Plantaren® (600 CS/U, 1200 and 2000) or Plantacare® (818, 1200 and 2000). Use may also be made of the products sold by the company SEPPIC under the names Triton CG 110 (or Oramix CG 110) and Triton CG 312 (or Oramix® NS 10), the products sold by the company BASF under the name Lutensol GD 70 or the products sold by the company Chem Y under the name AG10 LK, or the products sold by the company Evonik Goldschmidt under the trade names Tego Care CG 90 or Tego Care CG 90 MB.

The nonionic surfactant(s) of alkyl (poly)glycoside type present in the composition of the invention, are preferably chosen from caprylyl/capryl glucoside, decyl glucoside, coco glucoside, lauryl glucoside, myristyl glucoside, cetearyl glucoside, arachidyl glucoside and mixtures thereof. More preferentially, the nonionic surfactant(s) of alkyl (poly)glycoside type are chosen from caprylyl/capryl glucoside, decyl glucoside, coco glucoside, lauryl glucoside and mixtures thereof. Even more preferentially, the nonionic surfactant of alkyl (poly)glycoside type is coco glucoside.

Advantageously, the total content of the nonionic surfactant(s) of alkyl (poly)glycoside type present in the composition according to the invention is less than or equal to 3% by weight, preferably less than or equal to 2% by weight, and more preferentially less than or equal to 1% by weight, relative to the total weight of the composition.

The total content of the nonionic surfactant(s) of alkyl (poly)glycoside type present in the composition according to the invention may preferably range from 0.05% to 3% by weight, more preferentially from 0.07% to 2% by weight, and even better still from 0.1% to 1% by weight, relative to the total weight of the composition.

(iii) Polyglycerol Esters

The composition according to the present invention further comprises one or more polyglycerol esters, also referred to as polyglycerol esters nonionic surfactants. In other words, the polyglycerol ester(s) (iii) are different from the nonionic surfactants of alkyl (poly)glycoside type (ii) defined above.

The polyglycerol ester(s) (iii) according to the invention are preferably chosen from polyglycerol esters of a C₆-C₃₀ fatty acid.

The polyglycerol ester(s) (iii) used are preferably chosen from the compounds of formula (IV) below and mixtures thereof:

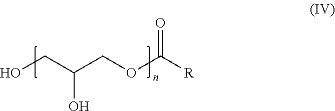

in which:
R represents a saturated or unsaturated, linear or branched, alkyl group comprising from 5 to 29 carbon atoms, preferably from 7 to 23 carbon atoms, more preferentially from 7 to 19 carbon atoms, and even better still from 8 to 15 carbon atoms, and
n represents an integer ranging from 2 to 20, preferably from 2 to 10, and even better still from 2 to 6;

More particularly, the polyglycerol ester(s) (iii) are chosen from polyglyceryl-4 isostearate, polyglyceryl-3 oleate, polyglyceryl-2 sesquioleate, diglyceryl monooleate, tetraglyceryl monooleate, polyglyceryl-3 caprate, polyglyceryl-4 caprate, polyglyceryl-2 laurate, polyglyceryl-5 laurate, polyglyceryl-10 laurate and mixtures thereof. Even better still, the polyglycerol ester is polyglyceryl-4 caprate.

The total content of the polyglycerol ester(s) (iii) present in the composition of the invention preferably ranges from 0.05% to 10% by weight, and more preferentially from 0.1% to 1% by weight, relative to the total weight of the composition.

The weight ratio (R) between the total content of nonionic surfactants of alkyl (poly)glycoside type (ii) and the total content of polyglycerol esters (iii) is less than or equal to 1. Preferably, this ratio is strictly less than 1, more preferentially it ranges from 0.1 to 1, and even better still from 0.1 to 0.8.

The composition according to the present invention may optionally further comprise one or more additional monoglycerol esters (or glycerol esters), also referred to as monoglycerol ester nonionic surfactants. In other words, these additional monoglycerol esters are different from the polyglycerol esters (iii) defined above.

Preferably, the additional monoglycerol ester(s) that can be used are chosen from the compounds of formula (V) below, and mixtures thereof:

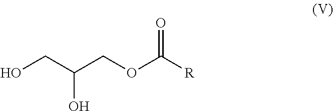

in which R represents a saturated or unsaturated, linear or branched, alkyl group comprising from 5 to 29 carbon atoms, preferably from 5 to 23 carbon atoms, more preferentially from 7 to 21 carbon atoms, and optionally comprising 1 or 2 hydroxyl groups (OH).

More preferentially, the additional monoglycerol ester(s) are chosen from glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl undecylenate and mixtures thereof.

The total content of the additional monoglycerol ester(s), when they are present in the composition of the invention, preferably ranges from 0.05% to 5% by weight and more preferentially from 0.1% to 1% by weight, relative to the total weight of the composition.

Preferably, the composition according to the invention comprises one or more polyglycerol esters (iii) and one or more additional monoglycerol esters. According to this variant, the total content of the (poly)glycerol ester(s) (i.e. the sum of the content of the polyglycerol ester(s) (iii) and the content of the additional monoglycerol ester(s)) preferably ranges from 0.1% to 10% by weight and more preferentially from 0.5% to 5% by weight, relative to the total weight of the composition.

According to this same variant, the weight ratio (R′) between the total content of alkyl (poly)glycoside nonionic surfactants (ii) and the total content of (poly)glycerol esters (also referred to as the content of monoglycerol and polyglycerol, i.e. the sum of the content of the polyglycerol ester(s) (iii) and the content of the additional monoglycerol ester(s)), present in the composition of the invention, is preferably less than or equal to 1, more preferentially less than or equal to 0.8, and even better still less than or equal to 0.6.

More preferentially, the weight ratio (R′) of the total content of alkyl (poly)glycoside nonionic surfactants (ii) to the total content of (poly)glycerol esters (also referred to as the content of monoglycerol and polyglycerol, i.e. the sum of the content of the polyglycerol ester(s) (iii) and the content of the additional monoglycerol ester(s)), present in the composition of the invention, ranges from 0.1 to 1, even more preferentially from 0.15 to 0.8, better from 0.2 to 0.6, and better still from 0.2 to 0.5.

The total content of nonionic surfactants (alkyl (poly)glycosides (ii), polyglycerol esters (iii), optionally additional monoglycerol esters, and optionally additional nonionic surfactants different from those mentioned above), present in the composition of the invention, preferably ranges from 0.05% to 10% by weight, and more preferentially from 0.1% to 5% by weight, relative to the total weight of the composition.

### (iv) Amphoteric Surfactants

The composition according to the present invention further comprises one or more amphoteric or zwitterionic surfactants.

In particular, the amphoteric or zwitterionic surfactant(s), which are preferably non-silicone, used in the composition according to the present invention may notably be derivatives of optionally quaternized aliphatic secondary or tertiary amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Mention may also be made, among the derivatives of optionally quaternized secondary or tertiary aliphatic amines which can be used, as defined above, of the compounds with the following respective structures (VI) and (VII):

Rₐ—CONHCH₂CH₂—N⁺(R_{b})(R_{c})—CH₂COO⁻,M⁺,X⁻ (VI)

in which formula (VI):
Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent;

Rₐ′—CONHCH₂CH₂—N(B)(B′) (VII)

in which formula (VII):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid Rₐ′—COOH which is preferably present in coconut kernel oil or in hydrolysed linseed oil, preferably Rₐ′ is an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (VIII):

Rₐ″—NHCH(Y″)—(CH₂)ₙCONH(CH₂)_{n′}—N(R_{d})(Rₑ) (VIII)

in which formula (VIII):
Y″ represents the group —COOH, —COOZ″ or —CH₂—CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid Rₐ″—COOH which is preferably present in coconut kernel oil or in hydrolysed linseed oil; and
n and n′ denote, independently of each other, an integer ranging from 1 to 3.

Mention may be made, among the compounds of formula (VIII), of the compound classified in the CTFA dictionary under the name sodium diethylaminopropyl cocoaspartamide and sold by Chimex under the name Chimexane HB.

These compounds may be used alone or as mixtures.

Among the amphoteric or zwitterionic surfactants mentioned above, use is advantageously made of (C₈-C₂₀)alkylbetaines, such as coco betaine, (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferentially, the amphoteric or zwitterionic surfactant(s) are chosen from (C₈-C₂₀)alkylbetaines, (C₈-C₂₀)alkylamido(C₃-C₈)alkylbetaines and mixtures thereof, even better still from (C₈-C₂₀)alkylamido(C₃-C₈)alkylbetaines and mixtures thereof.

The total content of the amphoteric or zwitterionic surfactant(s) present in the composition according to the invention preferably ranges from 0.5% to 10% by weight, more preferentially from 1% to 5% by weight, and even better still from 1% to 2.5% by weight, relative to the total weight of the composition.

### (v) Cationic Polymers

The composition according to the present invention also comprises one or more cationic polymers.

For the purposes of the present invention, the term “cationic polymer” means any polymer comprising cationic groups and/or groups that may be ionized to cationic groups. Preferably, the cationic polymer(s) are hydrophilic or amphiphilic.

The cationic polymers are not silicone-based (they do not comprise any Si—O units).

The cationic polymers may advantageously be chosen from associative cationic polymers, non-associative cationic polymers and mixtures thereof, and preferably from non-associative cationic polymers and mixtures thereof.

The preferred cationic polymers are chosen from those that contain units including primary, secondary, tertiary and/or quaternary amine groups that may either form part of the main polymer chain or may be borne by a side substituent directly connected thereto.

The cationic polymers according to the invention do not comprise any anionic groups or any groups that can be ionized to anionic groups.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:

(1) homopolymers or copolymers derived from acrylic or methacrylic esters or amides and including at least one of the units having the following formulae:

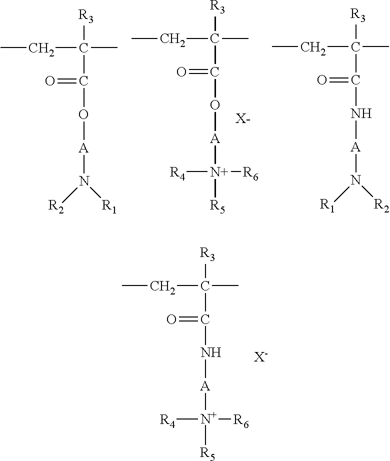

in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, preferably methyl or ethyl; and
X denotes an anion derived from a mineral or organic acid, such as a methosulfate anion or a halide such as chloride or bromide.

The copolymers of the family (1) may also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic acids or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by the company ISP;
polymers, preferably crosslinked polymers, of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.

(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethyl cellulose that have reacted with an epoxide substituted with a trimethylammonium group.

Cationic cellulose copolymers or cellulose derivatives grafted with a water-soluble quaternary ammonium monomer are described notably in U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

The cationic galactomannan gums are described more particularly in U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17 and Jaguar C162 by the company Rhodia.

(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.

(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyaminoamides can be alkylated or, if they include one or more tertiary amine functions, they can be quaternized;

(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.

(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or under the name PD 170 or Delsette 101 by the company Hercules in the case of the adipic acid/epoxypropyl/diethylenetriamine copolymer.

(7) Cyclopolymers of alkyldiallylamine or of dialkyldiallylammonium, such as the homopolymers or copolymers comprising, as main constituent of the chain, units corresponding to formula (IX) or (X):

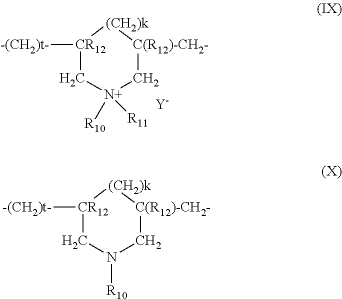

in which formulae (IX) and (X):
k and t are equal to 0 or 1, the sum k+t being equal to 1;
R₁₂ denotes a hydrogen atom or a methyl radical;
R₁₀ and R₁₁, independently of each other, denote an alkyl group containing from 1 to 6 carbon atoms, a hydroxyalkyl group in which the alkyl group contains 1 to 5 carbon atoms, a C₁ to C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold, for example, under the name Merquat 100 by the company Nalco (and homologues thereof of low weight-average molar masses) and the copolymers of diallyldimethylammonium salts (for example chloride) and of acrylamide, notably sold under the names Merquat 550 and Merquat 7SPR.

(8) quaternary diammonium polymers comprising repeating units of formula:

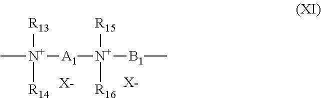

in which formula (XI):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;

it being understood that A₁, R₁₃ and R₁₅ can form, with the two nitrogen atoms to which they are attached, a piperazine ring;

in addition, if A₁ denotes a linear or branched, saturated or unsaturated alkylene or hydroxyalkylene radical, B₁ can also denote a group (CH₂)ₙCO-D-OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers which are constituted of repeating units corresponding to the formula:

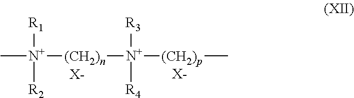

in which formula (XII) R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (XII) that is particularly preferred is the one for which R₁, R₂, R₃ and R₄ represent a methyl radical and n=3, p=6 and X=Cl, which is known as Hexadimethrine chloride according to the INCI (CTFA) nomenclature.

(9) polyquaternary ammonium polymers comprising units of formula (XIII):

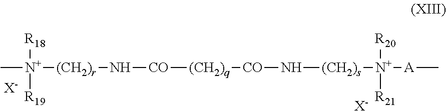

in which formula (XIII):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—

Examples that may be mentioned include the products Mirapol® A 15, Mirapol® AD1, Mirapol® AZ1 and Mirapol® 175 sold by the company Miranol.

(10) quaternary polymers of vinylpyrrolidone and of vinylimidazole, for instance the products sold under the names Luviquat® FC 905, FC 550 and FC 370 by the company BASF.

(11) polyamines such as Polyquart® H sold by Cognis, which is referenced under the name Polyethylene Glycol (15) Tallow Polyamine in the CTFA dictionary.

(12) polymers including in their structure:
(a) one or more units corresponding to formula (A) below:

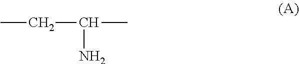

(b) optionally one or more units corresponding to formula (B) below:

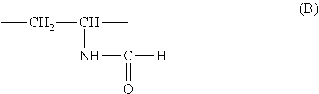

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The cationic charge density of these polymers may range from 2 meq/g to 20 meq/g, preferably from 2.5 to 15 meq/g and more particularly from 3.5 to 10 meq/g.

The polymers including units of formula (A) and optionally units of formula (B) are notably sold under the name Lupamin by the company BASF, for instance, in a non-limiting manner, the products sold under the names Lupamin 9095, Lupamin 5095, Lupamin 1095, Lupamin 9030 (or Luviquat 9030) and Lupamin 9010.

The cationic polymer(s) can be chosen from associative cationic polymers.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 8 carbon atoms, preferably from 8 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative cationic polymers that may be used, mention may be made, alone or as a mixture, of:
(A) cationic associative polyurethanes, which may be represented by the general formula (XIV) below:

R—X—(P)n-[L-(Y)m]r-L′-(P′)p—X′—R′

in which:
R and R′, which may be identical or different, represent a hydrophobic group or a hydrogen atom;
X and X′, which may be identical or different, represent a group comprising an amine function optionally bearing a hydrophobic group, or alternatively a group L″;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100 inclusive, preferably between 1 and 50 inclusive and in particular between 1 and 25 inclusive;
n, m and p are each, independently of one another, between 0 and 1000 inclusive;
the molecule containing at least one protonated or quaternized amine function and at least one hydrophobic group.

Preferably, the only hydrophobic groups are the groups R and R′ at the chain ends.

One preferred family of cationic associative polyurethanes is the one corresponding to formula (XIV) described above, in which:
R and R′ both independently represent a hydrophobic group,
X and X′ each represent a group L″,
n and p are integers that are between 1 and 1000 inclusive, and L, L′, L″, P, P′, Y and m have the meaning indicated above.

Another preferred family of cationic associative polyurethanes is the one corresponding to formula (XIV) above in which:
n=p=0 (the polymers do not include any units derived from a monomer containing an amine function, incorporated into the polymer during the polycondensation),
the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate.

Yet another preferred family of cationic associative polyurethanes is the one corresponding to formula (XIV) above in which:
R and R′ both independently represent a hydrophobic group,
X and X′ both independently represent a group including a quaternary amine,
n=p=0, and
L, L′, Y and m have the meaning given above.

The number-average molecular mass (Mn) of the cationic associative polyurethanes is preferably between 400 and 500 000 inclusive, in particular between 1000 and 400 000 inclusive and ideally between 1000 and 300 000 inclusive.

The term “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it includes at least 8 carbon atoms, preferably from 8 to 30 carbon atoms, in particular from 12 to 30 carbon atoms and more preferentially from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group including a tertiary or quaternary amine, X and/or X′ may represent one of the following formulae:

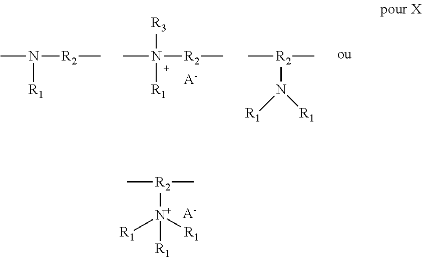

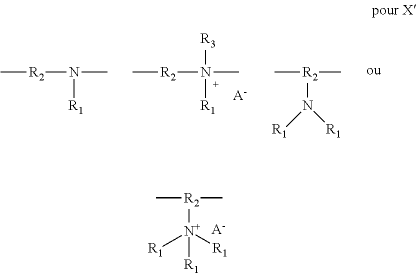

in which:
R₂ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P;
R₁ and R₃, which may be identical or different, denote a linear or branched C1-C30 alkyl or alkenyl radical or an aryl radical, at least one of the carbon atoms possibly being replaced with a heteroatom chosen from N, S, O and P;
A⁻ is a physiologically acceptable anionic counterion such as a halide, for instance a chloride or bromide, or a mesylate.

The groups L, L′ and L″ represent a group of formula:

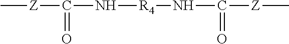

in which:
Z represents —O—, —S— or —NH—; and
R₄ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P.

The groups P and P′ comprising an amine function may represent at least one of the following formulae:

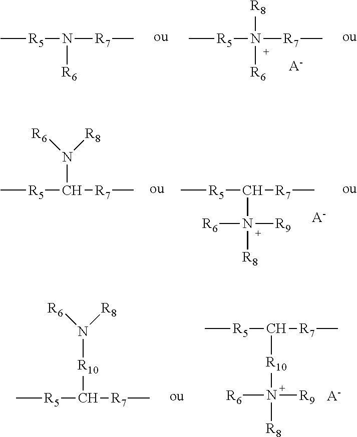

in which:
R₅ and R₇ have the same meanings as R2 defined previously;
R₆, R₈ and R₉ have the same meanings as R₁ and R₃ defined previously;
R₁₀ represents a linear or branched, optionally unsaturated alkylene group possibly containing one or more heteroatoms chosen from N, O, S and P;
and A⁻ is a physiologically acceptable anionic counterion such as a halide, for instance chloride or bromide, or mesylate.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and notably a poly(ethylene oxide) or poly(propylene oxide).

The cationic associative polyurethanes of formula (XIV) according to the invention are formed from diisocyanates and from various compounds bearing functions containing labile hydrogen. The functions containing labile hydrogen may be alcohol, primary or secondary amine or thiol functions, giving, after reaction with the diisocyanate functions, polyurethanes, polyureas and polythioureas, respectively. In the present invention, the term “polyurethanes” encompasses these three types of polymer, namely polyurethanes per se, polyureas and polythioureas, and also copolymers thereof.

A first type of compound involved in the preparation of the polyurethane of formula (XIV) is a compound comprising at least one unit containing an amine function. This compound may be multifunctional, but the compound is preferentially difunctional, that is to say that, according to a preferential embodiment, this compound includes two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may include more than one unit containing an amine function. In this case, it is a polymer bearing a repetition of the unit containing an amine function.

Compounds of this type may be represented by one of the following formulae:

HZ—(P)ₙ—ZH, or HZ—(P′)ₚ—ZH,

in which Z, P, P′, n and p are as defined above.

Examples that may be mentioned include N-methyldiethanolamine, N-tert-butyldiethanolamine and N-sulfoethyldiethanolamine.

The second compound included in the preparation of the polyurethane of formula (XIV) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (XIV) is a hydrophobic compound intended to form the terminal hydrophobic groups of the polymer of formula (XIV).

This compound is constituted of a hydrophobic group and a function containing a labile hydrogen, for example a hydroxyl, primary or secondary amine, or thiol function.

By way of example, this compound may be a fatty alcohol such as stearyl alcohol, dodecyl alcohol or decyl alcohol. When this compound includes a polymeric chain, it may be, for example, α-hydroxylated hydrogenated polybutadiene.

The hydrophobic group of the polyurethane of formula (XIV) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing labile hydrogen are alcohol, primary or secondary amine or thiol functions. This compound may be a polymer terminated at the chain ends with one of these functions containing labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and notably a poly(ethylene oxide) or poly(propylene oxide).

The hydrophilic group termed Y in formula (XIV) is optional. Specifically, the units containing a quaternary or protonated amine function may suffice to provide the solubility or water-dispersibility required for this type of polymer in an aqueous solution.

Although the presence of a hydrophilic group Y is optional, cationic associative polyurethanes including such a group are, however, preferred.
(B) quaternized cellulose derivatives, in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethyl celluloses of formula (XV):

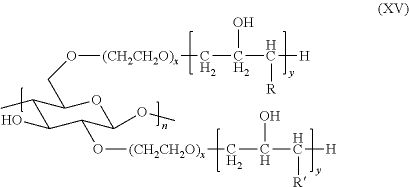

in which:
R represents an ammonium group RₐR_{b}R_{c}N⁺—, Q⁻ in which Rₐ, R_{b} and R_{c}, which may be identical or different, represent a hydrogen atom or a linear or branched C₁-C₃₀ alkyl, preferably an alkyl, and Q⁻ represents an anionic counterion such as a halide, for instance a chloride or bromide;
R′ represents an ammonium group R′ₐR′_{b}R′_{c}N⁺—, Q′⁻ in which R′ₐ, R′_{b} and R′_{c}, which may be identical or different, represent a hydrogen atom or a linear or branched C₁-C₃₀ alkyl, preferably an alkyl, and Q′⁻ represents an anionic counterion such as a halide, for instance a chloride or bromide;

it being understood that at least one of the radicals Rₐ, R_{b}, R_{c}, R′ₐ, R′_{b} and R′_{c} represents a linear or branched C₈-C30 alkyl;
n, x and y, which may be identical or different, represent an integer of between 1 and 10 000.

Preferably, in formula (XV), at least one of the radicals Rₐ, R_{b}, R_{c}, R′ₐ, R′_{b} or R′_{c} represents a linear or branched C₈ to C₃₀, better still C₁₀ to C₂₄, or even C₁₀ to C₁₄ alkyl; mention may be made in particular of the dodecyl radical (C₁₂). Preferably, the other radical(s) represent a linear or branched C₁-C₄ alkyl, notably methyl.

Preferably, in formula (XV), only one of the radicals Rₐ, R_{b}, R_{c}, R′ₐ, R′_{b} or R′_{c} represents a linear or branched C₈ to C₃₀, better still C₁₀ to C₂₄, or even C₁₀ to C₁₄ alkyl; mention may be made in particular of the dodecyl radical (C₁₂). Preferably, the other radicals represent a linear or branched C₁ to C₄ alkyl, notably methyl.

Even better still, R may be a group chosen from —N⁺(CH₃)₃, Q′⁻ and

—N⁺(C₁₂H₂₅)(CH₃)₂, Q′⁻, preferably a group —N⁺(CH₃)₃, Q′⁻.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (XV) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (XV) are, for example, those whose viscosity is between 2000 and 3000 cPs (2 Pa·s and 3 Pa·s) inclusive, preferentially between 2700 and 2800 cPs (2.7 Pa·s and 2.8 Pa·s). Typically, Softcat Polymer SL-5 has a viscosity of 2500 cPs (2.5 Pa·s), Softcat Polymer SL-30 has a viscosity of 2700 cPs, Softcat Polymer SL-60 has a viscosity of 2700 cPs (2.7 Pa·s) and Softcat Polymer SL-100 has a viscosity of 2800 cPs (2.8 Pa·s). Use may also be made of Softcat Polymer SX-1300X with a viscosity of between 1000 and 2000 cPs (between 1 Pa·s and 2 Pa·s).
(C) cationic polyvinyllactams, notably those comprising:
a) at least one monomer of vinyllactam or alkylvinyllactam type;
b) at least one monomer of structure (XVI) or (XVII) below:

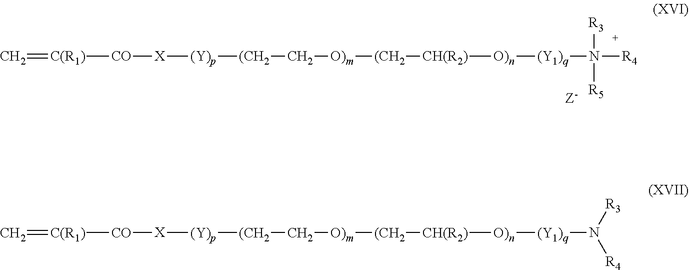

in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (XVIII):

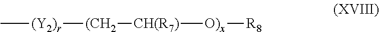

Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, 0 or 1,
m and n denote, independently of each other, an integer ranging from 0 to 100 inclusive,
x denotes an integer ranging from 1 to 100 inclusive,
Z denotes an anionic counterion of an organic or mineral acid, such as a halide, for instance chloride or bromide, or mesylate;

with the proviso that:
at least one of the substituents R₃, R₄, R₅ or R₈ denotes a linear or branched C₉-C₃₀ alkyl radical,
if m and/or n is other than zero, then q is equal to 1,
if m=n=0, then p or q is equal to 0.

The cationic poly(vinyllactam) polymers according to the invention may be crosslinked or non-crosslinked and may also be block polymers.

Preferably, the counterion Z⁻ of the monomers of formula (XVI) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferentially, the monomer b) is a monomer of formula (XVI) for which, preferentially, m and n are equal to 0.

The vinyllactam or alkylvinyllactam monomer is preferably a compound of structure (XIX):

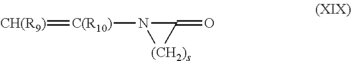

in which:
s denotes an integer ranging from 3 to 6,
R₉ denotes a hydrogen atom or a linear or branched C₁-C₅ alkyl radical,
R₁₀ denotes a hydrogen atom or a linear or branched C₁-C₅ alkyl radical,

with the proviso that at least one of the radicals R₉ and R₁₀ denotes a hydrogen atom.

Even more preferentially, the monomer (XIX) is vinylpyrrolidone.

The cationic poly(vinyllactam) polymers according to the invention may also contain one or more additional monomers, preferably cationic or nonionic monomers.

As compounds that are particularly preferred, mention may be made of the following terpolymers comprising at least:
a) one monomer of formula (XIX),
b) one monomer of formula (XIX) in which p=1, m=n=q=0, R₃ and R₄ denote, independently of each other, a hydrogen atom or a C₁-C₅ alkyl radical and R₅ denotes a linear or branched C₉-C₂₄ alkyl radical, and
c) one monomer of formula (XVII) in which p=1, m=n=q=0, R₃ and R₄ denote, independently of each other, a hydrogen atom or a linear or branched C₁-C₅ alkyl radical.

Even more preferentially, terpolymers comprising, by weight, 40% to 95% of monomer (a), 0.1% to 55% of monomer (c) and 0.25% to 50% of monomer (b) will be used. Such polymers are notably described in patent application WO-00/68282.

As cationic polymer(vinyllactam) polymers according to the invention, the following are notably used:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylmethylacrylamidopropylammonium chloride terpolymer is notably sold by the company ISP under the names Styleze W10® and Styleze W20L® (INCI name: Polyquaternium-55).

The weight-average molecular mass (Mw) of the cationic poly(vinyllactam) polymers is preferably between 500 and 20 000 000, more particularly between 200 000 and 2 000 000 and preferentially between 400 000 and 800 000.
(D) the cationic polymer(s) obtained by polymerization of a monomer mixture comprising one or more vinyl monomers substituted with one or more amino groups, one or more hydrophobic nonionic vinyl monomers, and one or more associative vinyl monomers, as described in patent application WO 2004/024779.

Among these copolymers, mention may be made more particularly of the products of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

Such a polymer is, for example, the compound sold by the company Lubrizol under the name Carbopol Aqua CC® and which corresponds to the INCI name Polyacrylate-1 Crosspolymer.

Advantageously, the associative cationic polymer(s) are chosen from quaternized (poly)hydroxyethylcelluloses modified with groups including at least one fatty chain.

Advantageously, the cationic polymer(s) are chosen from non-associative cationic polymers and mixtures thereof, preferably from cationic polysaccharides (the family (2)) and mixtures thereof, more preferentially from cationic galactomannan gums and mixtures thereof, and even better still from cationic guar gums and mixtures thereof.

The total content of the cationic polymer(s) (v), present in the composition according to the invention, preferably ranges from 0.05% to 2% by weight, and more preferentially from 0.1% to 1% by weight, relative to the total weight of the composition.

### Thickening Polymers

The composition according to the present invention may optionally also comprise one or more thickening polymers, different from the cationic polymer(s) (v) defined above.

According to the present invention, the term “thickening polymers” means polymers which, by their presence at a concentration of 0.05% by weight, increase the viscosity of the cosmetic compositions into which they are introduced by at least 20 cps (20 mPa·s), preferably by at least 50 cps (50 mPa·s), at room temperature (25° C.), at atmospheric pressure and at a shear rate of 1 s⁻¹ (the viscosity may be measured using a cone/plate viscometer, a Haake R600 rheometer or the like).

The thickening polymer(s) that can be used according to the invention are different from the cationic polymer(s) (v) defined above, they are therefore anionic, amphoteric or nonionic; they are preferentially chosen from non-associative anionic, amphoteric or nonionic thickening polymers bearing sugar units, non-associative anionic, amphoteric or nonionic thickening polymers without sugar units, associative anionic, amphoteric or nonionic thickening polymers, and mixtures thereof.

For the purposes of the present invention, the term “sugar unit” means an oxygen-bearing hydrocarbon-based compound containing several alcohol functions, with or without aldehyde or ketone functions, and which includes at least 4 carbon atoms.

The sugar units may be optionally modified by substitution, and/or by oxidation and/or by dehydration.

The sugar units of the thickening polymers are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

According to the invention, polymers bearing sugar units are also known as polysaccharides.

Mention may in particular be made, as non-associative thickening polymers bearing sugar units, of native gums, such as:
a) tree or shrub exudates, including:
   acacia gum (branched polymer of galactose, arabinose, rhamnose and glucuronic acid);
   ghatti gum (polymer derived from arabinose, galactose, mannose, xylose and glucuronic acid);
   karaya gum (polymer derived from galacturonic acid, galactose, rhamnose and glucuronic acid);
   gum tragacanth (polymer of galacturonic acid, galactose, fucose, xylose and arabinose);
b) gums derived from algae, including:
   agar (polymer derived from galactose and anhydrogalactose);
   alginates (polymers of mannuronic acid and of glucuronic acid);
   carrageenans and furcellerans (polymers of galactose sulfate and of anhydrogalactose sulfate);
c) gums derived from seeds or tubers, including:
   guar gum (polymer of mannose and galactose);
   locust bean gum (polymer of mannose and galactose),
   fenugreek gum (polymer of mannose and galactose);
   tamarind gum (polymer of galactose, xylose and glucose);
   konjac gum (polymer of glucose and mannose);
d) microbial gums, including:
   xanthan gum (polymer of glucose, mannose acetate, mannose/pyruvic acid and glucuronic acid);
   gellan gum (polymer of partially acylated glucose, rhamnose and glucuronic acid);
   scleroglucan gum (glucose polymer);
e) plant extracts, including:
   cellulose (glucose polymer);
   starch (glucose polymer) and
   inulin.

These polymers may be physically or chemically modified. As physical treatment, mention may notably be made of the temperature.

Mention may be made, as chemical treatments, of esterification, etherification, amidation or oxidation reactions. These treatments make it possible to lead to polymers that may in particular be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly)hydroxyalkyl groups.

Among the C₁-C₆ (poly)hydroxyalkyl groups, mention may be made, by way of example, of hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by Rhodia Chimie.

The botanical origin of the starch molecules that can be used in the present invention may be cereals or tubers. Thus, the starches are chosen, for example, from corn starch, rice starch, cassava starch, barley starch, potato starch, wheat starch, sorghum starch or pea starch.

The starches can be chemically or physically modified, in particular by one or more of the following reactions: pregelatinization, oxidation, crosslinking, esterification, etherification, amidation or heat treatments.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetylated cassava distarch phosphate) by the company Avebe, or Structure Zea from National Starch (gelatinized maize distarch phosphate).

According to the invention, use may also be made of amphoteric starches, these amphoteric starches comprising one or more anionic groups and one or more cationic groups. The anionic and cationic groups can be bonded to the same reactive site of the starch molecule or to different reactive sites; they are preferably bonded to the same reactive site. The anionic groups may be of carboxylic, phosphate or sulfate type, preferably carboxylic type. The cationic groups may be of primary, secondary, tertiary or quaternary amine type.

The starch molecules may be derived from any plant source of starch, in particular such as corn, potato, oat, rice, tapioca, sorghum, barley or wheat. It is also possible to use hydrolyzates of the starches mentioned above. The starch is preferably derived from potato.

The non-associative thickening polymers that can be used according to the invention may be cellulose polymers not comprising a C₁₀-C₃₀ fatty chain in their structure.

According to the invention, the term “cellulose-based polymer” means any polysaccharide compound having in its structure sequences of glucose residues linked together via β-1,4 bonds; in addition to unsubstituted celluloses, the cellulose derivatives may be anionic, amphoteric or nonionic.

Thus, the cellulose-based polymers that can be used according to the invention may be chosen from unsubstituted celluloses, including those in a microcrystalline form, and cellulose ethers.

Among these cellulose-based polymers, cellulose ethers, cellulose esters and cellulose ether esters are distinguished.

Among the cellulose esters are inorganic esters of cellulose (cellulose nitrates, sulfates, phosphates, etc.), organic esters of cellulose (cellulose monoacetates, triacetates, amidopropionates, acetatebutyrates, acetatepropionates or acetatetrimellitates, etc.), and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers not bearing a C₁₀-C₃₀ fatty chain, i.e. which are “non-associative”, mention may be made of (C1-C4)alkylcelluloses, such as methylcelluloses and ethylcelluloses (for example, Ethocel Standard 100 Premium from Dow Chemical); (poly)hydroxy(C1-C4)alkylcelluloses, such as hydroxymethylcelluloses, hydroxyethylcelluloses (for example, Natrosol 250 HHR sold by Aqualon) and hydroxypropylcelluloses (for example, Klucel EF from Aqualon); mixed (poly)hydroxy(C1-C4)alkyl-(C1-C4)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly)carboxy(C1-C4)alkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and the sodium salts thereof.

Among the non-associative thickening polymers not bearing sugar units that may be used according to the invention, mention may be made of crosslinked acrylic acid or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or as mixtures.

A first family of non-associative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by the company Noveon or the products sold under the names Synthalen M and Synthalen K by the company 3 VSA. These polymers have the INCI name Carbomer.

The non-associative thickening polymers may also be crosslinked (meth)acrylic acid copolymers, such as the polymer sold under the name Aqua SF1 by the company Noveon.

The non-associative thickening polymers may be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of EP 503 853, and reference may be made to said document as regards these polymers.

The cosmetic composition may similarly comprise, as non-associative thickening polymers, ammonium acrylate homopolymers or copolymers of ammonium acrylate and of acrylamide.

As examples of ammonium acrylate homopolymers, mention may be made of the product sold under the name Microsap PAS 5193 by the company Hoechst. Among the copolymers of ammonium acrylate and of acrylamide, mention may be made of the product sold under the name Bozepol C Nouveau or the product PAS 5193 sold by the company Hoechst. Reference may notably be made to FR 2 416 723, U.S. Pat. Nos. 2,798,053 and 2,923,692 as regards the description and preparation of such compounds.

Among the thickening polymers that can be used according to the invention, mention may also be made of associative polymers that are well known to those skilled in the art and especially of nonionic, anionic or amphoteric nature.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type that can be used, mention may be made of:
(a) those including at least one hydrophilic unit and at least one fatty-chain allyl ether unit, more particularly those whose hydrophilic unit is constituted of an ethylenic unsaturated anionic monomer, more particularly still of a vinylcarboxylic acid and most particularly of an acrylic acid or a methacrylic acid or mixtures thereof.

Among these anionic associative polymers, the ones that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), notably those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10);
(b) those including i) at least one hydrophilic unit of unsaturated olefinic carboxylic acid type, and ii) at least one hydrophobic unit of the (C₁₀-C₃₀) alkyl ester of an unsaturated carboxylic acid type.

(C₁₀-C₃₀) Alkyl esters of unsaturated carboxylic acids that are useful in the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the anionic associative polymers of this type that will be used more particularly are those constituted of from 60% to 95% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those constituted of from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among said polymers above, the ones most particularly preferred according to the present invention are the products sold by the company Goodrich under the trade names Pemulen TR1®, Pemulen TR2®, Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP;
(c) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.
(d) acrylic terpolymers comprising:
i) about 20% to 70% by weight of an α,β-monoethylenically unsaturated carboxylic acid [A],
ii) about 20% to 80% by weight of an α,β-monoethylenically unsaturated non-surfactant monomer other than [A],
iii) about 0.5% to 60% by weight of a nonionic monourethane which is the product of reaction of a monohydric surfactant with a monoethylenically unsaturated monoisocyanate,
such as those described in patent application EP-A-0 173 109 and more particularly the one described in Example 3, namely a methacrylic acid/methyl acrylate/behenyl alcohol dimethyl-meta-isopropenylbenzylisocyanate ethoxylated (40 EO) terpolymer, as an aqueous 25% dispersion;
(e) copolymers including among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas.
(f) amphiphilic polymers including at least one ethylenically unsaturated monomer bearing a sulfonic group, in free or partially or totally neutralized form and comprising at least one hydrophobic part. These polymers may be crosslinked or non-crosslinked. They are preferably crosslinked.

The ethylenically unsaturated monomers bearing a sulfonic group are notably chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used.

2-Acrylamido-2-methylpropanesulfonic acid (AMPS), and also partially or totally neutralized forms thereof, will more particularly be used.

The polymers of this family may be chosen notably from random amphiphilic AMPS polymers modified by reaction with a C₆-C₂₂ n-monoalkylamine or di-n-alkylamine, and such as those described in patent application WO 00/31154 (forming an integral part of the content of the description). These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not including a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described notably in patent application EP-A 750 899, U.S. Pat. No. 5,089,578 and in the following publications from Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, Vol. 33, No. 10, (2000), 3694-3704;
Solution properties of micelle networks formed by non-ionic moieties covalently bound to an polyelectrolyte: salt effects on rheological behavior—Langmuir, Vol. 16, No. 12, (2000) 5324-5332;
Stimuli responsive amphiphilic copolymers of sodium 2-(acrylamido)-2-methylpropanesulfonate and associative macromonomers, Polym. Preprint, Div. Polym. Chem., 40(2), (1999), 220-221.

Among these polymers, mention may be made of:
crosslinked or non-crosslinked, neutralized or non-neutralized copolymers, including from 15% to 60% by weight of AMPS units and from 40% to 85% by weight of (C₈-C₁₆)alkyl(meth)acrylamide or (C₈-C₁₆)alkyl(meth)acrylate units relative to the polymer, such as those described in patent application EP-A750 899;
terpolymers including from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

The amphoteric associative polymers are preferably chosen from those including at least one noncyclic cationic unit. Even more particularly, those prepared from or comprising 1 to 20 mol %, preferably 1.5 to 15 mol % and even more particularly 1.5 to 6 mol % of fatty-chain monomer relative to the total number of moles of monomers are preferred.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:
(a) copolymers of vinylpyrrolidone and of fatty-chain hydrophobic monomers, of which examples that may be mentioned include:
   the products Antaron V216® or Ganex V216® (vinylpyrrolidone/hexadecene copolymer) sold by the company ISP,
   the products Antaron V220® or Ganex V220® (vinylpyrrolidone/eicosene copolymer) sold by the company ISP,
(b) copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name Antil 208®;
(c) copolymers of hydrophilic methacrylates or acrylates and of hydrophobic monomers including at least one fatty chain, for instance the polyethylene glycol methacrylate/lauryl methacrylate copolymer;
(d) polyurethane polyethers including in their chain both hydrophilic blocks usually of polyoxyethylenated nature and hydrophobic blocks, which may be aliphatic sequences alone and/or cycloaliphatic and/or aromatic sequences;
(e) polymers with an aminoplast ether backbone containing at least one fatty chain, such as the Pure Thix® compounds sold by the company Sud-Chemie;
(f) celluloses or derivatives thereof, modified with groups including at least one fatty chain, such as alkyl, arylalkyl or alkylaryl groups or mixtures thereof in which the alkyl groups have at least 8 carbon atoms, and in particular:
   nonionic alkylhydroxyethylcelluloses, such as the products Natrosol Plus Grade 330 CS and Polysurf 67 (C16 alkyl) sold by Aqualon;
   nonionic nonoxynyl hydroxyethylcelluloses, such as the product Amercell HM-1500 sold by Amerchol;
   nonionic alkylcelluloses, such as the product Bermocoll EHM 100 sold by the company Berol Nobel;
(g) associative guar derivatives, for instance hydroxypropyl guars modified with a fatty chain, such as the product Esaflor HM 22 (modified with a C22 alkyl chain) sold by the company Lamberti; the product Miracare XC 95-3 (modified with a C₁₄ alkyl chain) and the product RE 205-146 (modified with a C20 alkyl chain) sold by Rhodia Chimie.

Preferably, the polyurethane polyethers include at least two hydrocarbon-based lipophilic chains containing from 8 to 30 carbon atoms, separated by a hydrophilic block, the hydrocarbon-based chains possibly being side chains or chains at the end of the hydrophilic block. In particular, it is possible for one or more side chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The fatty-chain nonionic polyurethane polyethers may be triblock copolymers, the hydrophilic block of which is a polyoxyethylenated chain including from 50 to 1000 oxyethylene groups. The nonionic polyurethane polyethers include a urethane bond between the hydrophilic blocks, whence the origin of the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, use may also be made of Rheolate 205® bearing a urea function, sold by the company Rheox, or Rheolate® 208, 204 or 212, and also Acrysol RM 184®.

Mention may also be made of the product Elfacos T210® containing a C₁₂-C₁₄ alkyl chain, and the product Elfacos T212® containing a C₁₈ alkyl chain, from Akzo.

The product DW 1206B® from Röhm & Haas bearing a C₂₀ alkyl chain and a urethane bond, sold at a solids content of 20% in water, may also be used.

Use may also be made of solutions or dispersions of these polymers, notably in water or in aqueous-alcoholic medium. Examples of such polymers that may be mentioned are Rheolate® 255, Rheolate® 278 and Rheolate® 244 sold by the company Rheox. Use may also be made of the products DW 1206F and DW 1206J sold by the company Röhm & Haas.

The polyurethane polyethers that may be used according to the invention are in particular those described in the article by G. Fonnum, J. Bakke and Fk. Hansen—Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold notably by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

The thickening polymer(s) are preferably chosen from non-associative thickening polymers without sugar units and mixtures thereof, and more preferentially from crosslinked acrylic acid homopolymers and mixtures thereof.

The total content of the thickening polymer(s), when they are present in the composition of the invention, preferably ranges from 0.01% to 2% by weight and more preferentially from 0.05% to 1% by weight, relative to the total weight of the composition.

### The Organic Solvents

The composition according to the present invention may optionally also comprise one or more organic solvents.

Preferably, the organic solvent(s) are chosen from linear or branched monoalcohols having from 1 to 8 carbon atoms and more preferentially from 1 to 4 carbon atoms, polyols, polyethylene glycols, aromatic alcohols and mixtures thereof.

As examples of organic solvents that can be used according to the invention, mention may notably be made of ethanol, propanol, butanol, isopropanol, isobutanol, propylene glycol, dipropylene glycol, isoprene glycol, butylene glycol, glycerol, sorbitol, benzyl alcohol and phenoxyethanol, and mixtures thereof.

The organic solvent(s) that can be used according to the invention may be chosen from linear or branched monoalcohols containing from 1 to 4 carbon atoms, and mixtures thereof, preferably from ethanol, propanol, butanol, isopropanol, isobutanol, and mixtures thereof.

Preferably, the organic solvent(s) are chosen from polyols and mixtures thereof, and more preferentially, the organic solvent is glycerol.

In a preferred embodiment of the invention, the composition comprises one or more organic solvents, preferentially one or more polyols and even better still glycerol.

The total content of the organic solvent(s), when they are present in the composition according to the invention, preferably ranges from 0.1% to 10% by weight, and more preferentially from 0.5% to 5% by weight, relative to the total weight of the composition.

In a preferred variant of the invention, the composition further comprises glycerol, the total content of which ranges preferably from 0.1% to 10% by weight, and more preferentially from 0.5% to 5% by weight, relative to the total weight of the composition.

The composition according to the present invention comprises water.

The total content of water ranges from 60% to 99% by weight, more preferentially from 70% to 98% by weight, even better still from 75% to 95% by weight, and even more preferentially from 80% to 90% by weight, relative to the total weight of the composition.

The composition according to the present invention may optionally also comprise one or more additional compounds different from the compounds defined above, preferably chosen from nonionic surfactants different from the nonionic surfactants of alkyl (poly)glycoside type, (poly)glycerol esters defined above, cationic surfactants, anionic, nonionic and amphoteric polymers different from the thickening polymers defined previously, antioxidants, penetrants, sequestrants, fragrances, buffers, dispersants, conditioning agents such as for example modified or unmodified volatile or non-volatile silicones, film-forming agents, ceramides, preserving agents, opacifiers, lubricants (or anticaking agents) and mixtures thereof.

Preferably, when the above additional compound(s) are present in the composition according to the invention, the additional compound(s) are generally present in an amount, for each of them, of between 0.01% and 20% by weight, relative to the weight of the composition.

Needless to say, a person skilled in the art will take care to select this or these optional additional compound(s) such that the advantageous properties intrinsically associated with the composition of the invention are not, or are not substantially, adversely affected by the envisaged addition(s).

The pH of the composition according to the invention generally ranges from 3 to 9, preferably from 4 to 6.

The pH of the composition may be adjusted to the desired value by means of the basifying agents or acidifying agents that are customarily used. Among the basifying agents, examples that may be mentioned include aqueous ammonia, alkanolamines, and mineral or organic hydroxides. Among the acidifying agents, examples which may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

Another subject of the present invention is a method for cosmetic treatment, in particular for washing and/or conditioning, of keratin fibres, notably human keratin fibres such as the hair, comprising the application to said keratin fibres of a composition as defined previously; this application optionally being followed by rinsing after an optional leave-on time.

Preferably, the application of the composition according to the invention is followed by rinsing.

The composition may be applied to wet or dry keratin fibres. It is preferably applied to wet keratin materials. It is advantageously left in place on the keratin fibres for a time ranging from 1 to 15 minutes, preferably from 2 to 10 minutes.

At the end of the process, the keratin fibres may optionally be dried using a hairdryer or a hood or left to dry naturally.

Another subject of the present invention is the use of a composition as defined previously for washing and/or conditioning keratin fibres, and in particular human keratin fibres such as the hair.

The examples that follow serve to illustrate the invention without, however, being limiting in nature.

### EXAMPLES

### I. Example 1

### 1. The Compositions Tested

The following compositions A1 and A2 according to the invention were prepared from ingredients, the contents of which are indicated in the tables below (% in g of active material).

| | A1 | A2 |
|---|---|---|
| Sodium lauryl sulfate | 7.5 | 7.0 |
| Polyglyceryl-4 caprate | 0.4 | 0.4 |
| Cocobetaine | 1.5 | 1.5 |
| Glyceryl oleate | 0.4 | 0.4 |
| Hydroxypropyl guar | 0.4 | 0.4 |
| hydroxypropyltrimonium chloride | | |
| Coco-glucoside | 0.25 | 0.25 |
| Sodium chloride | 0.8 | 0.8 |
| Glycol distearate | 0.5 | 0.5 |
| Triethyl citrate | 0.5 | 0.5 |
| Glycerol | 3 | — |
| Preserving agents | qs | qs |
| Carbomer | 0.1 | 0.1 |
| Water | qs 100 | qs 100 |
| APG/polyglycerol ester weight ratio | 0.625 | 0.625 |
| APG/(poly)glycerol ester weight | 0.3 | 0.3 |
| ratio | | |

Compositions A1 and A2 thus obtained may be used as shampoo. They exhibit good foam properties (fine, soft and creamy foam) and give the hair a good level of conditioning. The hair is notably more manageable, smoother and easier to disentangle, whether on wet hair or on dry hair. The compositions further impart a feeling of freshness and a natural and non-greasy feel to the hair, which lasts over time.

### II. Example 2

### 1. Composition Tested

The following shampoo composition A3 according to the invention was prepared from ingredients, the contents of which are indicated in the tables below (% in g of active material).

| | A3 |
|---|---|
| Sodium lauryl sulfate | 7.54 |
| Polyglyceryl-4 caprate | 0.43 |
| Cocobetaine | 1.5 |
| Glyceryl oleate | 0.42 |
| Hydroxypropyl guar | 0.38 |
| hydroxypropyltrimonium chloride | |
| Coco-glucoside | 0.26 |
| Sodium chloride | 0.8 |
| Glycol distearate | 0.5 |
| Triethyl citrate | 0.5 |
| Glycerol | 3 |
| Preserving agents | qs |
| Carbomer | 0.1 |
| Water | qs 100 |
| APG/polyglycerol ester weight ratio | 0.6 |

### 2. Protocol

The viscosity of composition A3 was measured with a Ford cup 10 (25° C., 1 atm), directly after obtaining the composition, then after 2 months of storage at 37° C. and 45° C.

The appearance and the homogeneity of the composition were also evaluated visually.

### 3. Results

The results are expressed in the table below.

| | Viscosity | | |
|---|---|---|---|
| | (Pa · s) | pH | Appearance |
| At T₀ | 6.2 | 5.4 | Opaque and |
| | | | homogeneous |
| After 2 months of | 6.2 | 5.4 | Opaque and |
| storage at 37° C. | | | homogeneous |
| After 2 months of | 6.2 | 5.4 | Opaque and |
| storage at 45° C. | | | homogeneous |

The results obtained above show that the composition of the invention remains stable over time, even after two months of storage at 45° C.

### III. Example 3

### 1. The Compositions Tested

The following comparative compositions B1 and B2 were prepared from ingredients, the contents of which are indicated in the tables below (% in g of active material).

The comparative compositions B1 and B2 were then compared with the composition A1 according to the invention from Example 1.

| | A1 | B1 | B2 |
|---|---|---|---|
| Sodium lauryl sulfate | 7.5 | 7.5 | 7.5 |
| Polyglyceryl-4 caprate | 0.4 | 0.4 | 0.165 |
| Cocobetaine | 1.5 | 1.5 | 1.5 |
| Glyceryl oleate | 0.4 | 0.4 | 0.4 |
| Hydroxypropyl guar | 0.4 | 0.4 | 0.4 |
| hydroxypropyltrimonium | | | |
| chloride | | | |
| Coco-glucoside | 0.25 | 0.6 | 0.25 |
| Sodium chloride | 0.8 | 0.8 | 0.8 |
| Glycol distearate | 0.5 | 0.5 | 0.5 |
| Triethyl citrate | 0.5 | 0.5 | 0.5 |
| Glycerol | 3 | 3 | 3 |
| Preserving agents | qs | qs | qs |
| Carbomer | 0.1 | 0.1 | 0.1 |
| Water | qs 100 | qs 100 | qs 100 |
| APG/polyglycerol ester | 0.625 | 1.50 | 1.51 |
| weight ratio | | | |

### 2. Protocol

The compositions A1, B1 or B2 are applied in a standardized way to moderately sensitized locks (alkaline solubility=20%, SA 20) previously humidified, by kneading the locks for 15 sec. (6 passages between the fingers), at a rate of 1 g of composition per 2.7 grams of hair.

After a 1 min pause, the hair is rinsed for 20 sec. (25 passages between the fingers).

The hair is then wrung and evaluated on the criteria of smoothness (to the touch) and suppleness (both on wet hair). These evaluations are carried out by 5 expert evaluators, who give a score ranging from 0 (very bad on the criterion) to 5 (very good on the criterion), in steps of 0.5.

### 3. Results

| | A1 | B1 | B2 |
|---|---|---|---|
| Smoothness | (Invention) | (Comparative) | (Comparative) |
| Expert 1 | 4 | 3 | 2 |
| Expert 2 | 4 | 3 | 2 |
| Expert 3 | 4.5 | 3.5 | 3.5 |
| Expert 4 | 4 | 4 | 2 |
| Expert 5 | 4.5 | 3.5 | 2.5 |
| Average | 4.2 | 3.4 | 2.4 |
| Standard deviation | 0.3 | 0.4 | 0.7 |

| | A1 | B1 | B2 |
|---|---|---|---|
| Suppleness | (Invention) | (Comparative) | (Comparative) |
| Expert 1 | 4 | 3 | 2 |
| Expert 2 | 4 | 2 | 3 |
| Expert 3 | 4.5 | 4 | 4 |
| Expert 4 | 4 | 3 | 3 |
| Expert 5 | 4.5 | 3 | 2 |
| Average | 4.2 | 3 | 2.8 |
| Standard deviation | 0.3 | 0.7 | 0.8 |

It is noted that the locks treated with composition A1 according to the invention are significantly better on the criteria of smoothness and suppleness, compared with the locks treated with the comparative compositions B1 and B2.

1. Composition comprising:
(i) one or more anionic surfactants,
(ii) one or more nonionic surfactants of alkyl (poly)glycoside type,
(iii) one or more polyglycerol esters,
(iv) one or more amphoteric or zwitterionic surfactants, and
(v) one or more cationic polymers;
wherein the total content of water ranges from 60% to 99% by weight, relative to the total weight of the composition, and
the weight ratio between the total content of nonionic surfactants of alkyl (poly)glycoside type (ii) and the total content of polyglycerol esters (iii) is less than or equal to 1.

2. Composition according to claim 1, characterized in that the anionic surfactant(s) (i) are chosen from sulfate-type anionic surfactants which are preferably not oxyalkylenated, and mixtures thereof, and more preferentially from alkyl sulfates, alkyl ether sulfates, alkylamido sulfates, alkylamido ether sulfates, alkylaryl polyether sulfates, monoglyceride sulfates; salts thereof, and mixtures thereof; the alkyl groups of these compounds notably including from 8 to 30 carbon atoms, preferably from 8 to 26, and more preferentially from 10 to 22 carbon atoms; the aryl group preferably denoting a phenyl or benzyl group.

3. Composition according to claim 1, characterized in that the anionic surfactant(s) (i) are chosen from:
alkyl sulfates, notably C₈ to C₂₆, and preferably C₁₀ to C₂₂, alkyl sulfates;
alkyl ether sulfates, notably C₈ to C₂₆ and preferably C₁₀ to C₂₂ alkyl ether sulfates, preferably comprising from 1 to 10 ethylene oxide units;
in particular in the form of alkali metal, alkaline-earth metal, ammonium or amino alcohol salts; and
mixtures thereof; and
preferably chosen from sodium, triethanolamine, magnesium or ammonium (C₁₀-C₂₂)alkyl sulfates, sodium, ammonium or magnesium (C₁₀-C₂₂)alkyl ether sulfates, and mixtures thereof.

4. Composition according to claim 1, characterized in that the content of the anionic surfactant(s) (i) ranges from 1% to 30% by weight, and preferably from 2% to 20% by weight, and more preferentially from 5% to 10% by weight, relative to the total weight of the composition.

5. Composition according to claim 1, characterized in that the alkyl (poly)glycoside nonionic surfactant(s) (ii) are chosen from compounds of formula (III) below and mixtures thereof:
R₁O—(R₂O)ₜ(G)ᵥ (III)
in which formula (III):
R₁ represents a linear or branched, saturated or unsaturated alkyl group including from 6 to 24 carbon atoms, or an alkylphenyl group in which the linear or branched alkyl group includes from 6 to 24 carbon atoms,
R₂ represents an alkylene group including from about 2 to 4 carbon atoms,
G represents a saccharide unit including 5 or 6 carbon atoms,
t denotes a value ranging from 0 to 10 and preferably 0 to 4, and
v denotes a value ranging from 1 to 15;
preferably chosen from caprylyl/capryl glucoside, decyl glucoside, coco glucoside, lauryl glucoside, myristyl glucoside, cetearyl glucoside, arachidyl glucoside and mixtures thereof; and more preferentially, the nonionic surfactant of alkyl (poly)glycoside type is coco glucoside.

6. Composition according to claim 1, characterized in that the total content of the nonionic surfactant(s) of alkyl(poly)glycoside type (ii) ranges from 0.05% to 3% by weight, preferably from 0.07% to 2% by weight, and more preferentially from 0.1% to 1% by weight, relative to the total weight of the composition.

7. Composition according to claim 1, characterized in that the polyglycerol ester(s) (iii) are chosen from the polyglycerol esters of a C₆ to C₃₀ fatty acid.

8. Composition according to claim 1, characterized in that the polyglycerol ester(s) (iii) are chosen from the compounds of formula (IV) below and mixtures thereof: 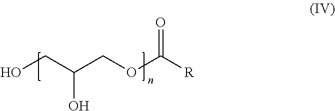
in which:
R represents a saturated or unsaturated, linear or branched, alkyl group comprising from 5 to 29 carbon atoms, preferably from 7 to 23 carbon atoms, more preferentially from 7 to 19 carbon atoms, and even better still from 8 to 15 carbon atoms, and
n represents an integer ranging from 2 to 20, preferably from 2 to 10, and even better still from 2 to 6;
preferably from polyglyceryl-4 isostearate, polyglyceryl-3 oleate, polyglyceryl-2 sesquioleate, diglyceryl monooleate, tetraglyceryl monooleate, polyglyceryl-3 caprate, polyglyceryl-4 caprate, polyglyceryl-2 laurate, polyglyceryl-5 laurate, polyglyceryl-10 laurate and mixtures thereof; and more preferentially, the polyglycerol ester is polyglyceryl-4 caprate.

9. Composition according to claim 1, characterized in that the total content of the nonionic surfactant(s) of polyglycerol ester type (iii) ranges from 0.05% to 5% by weight, and preferably from 0.1% to 1% by weight, relative to the total weight of the composition.

10. Composition according to claim 1, characterized in that the weight ratio between the total content of nonionic surfactants of alkyl (poly)glycoside type (ii) and the total content of polyglycerol esters (iii) ranges from 0.1 to 1, and preferably from 0.1 to 0.8.

11. Composition according to claim 1, characterized in that it further comprises one or more additional monoglycerol esters, different from the polyglycerol esters (iii), preferably chosen from glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl undecylenate and mixtures thereof.

12. Composition according to claim 11, characterized in that the total content of the additional monoglycerol ester(s) ranges from 0.05% to 5% by weight, and preferably from 0.1% to 1% by weight, relative to the total weight of the composition.

13. Composition according to claim 11, characterized in that the weight ratio of the total content of alkyl (poly)glycoside nonionic surfactants (ii) to the total content of (poly)glycerol esters, ranges from 0.1 to 1; preferably from 0.15 to 0.8; more preferentially from 0.2 to 0.6; and even more preferentially from 0.2 to 0.5.

14. Composition according to claim 1, characterized in that the amphoteric or zwitterionic surfactant(s) (iv) are chosen from (C₈-C₂₀)alkylbetaines, (C₈-C₂₀)alkylamido(C₃-C₈)alkylbetaines and mixtures thereof, and preferably from (C₈-C₂₀)alkylamido(C₃-C₈)alkylbetaines and mixtures thereof.

15. Composition according to claim 1, characterized in that the total content of the amphoteric or zwitterionic surfactant(s) (iv) ranges from 0.5% to 10% by weight, preferably from 1% to 5% by weight, and more preferentially from 1% to 2.5% by weight, relative to the total weight of the composition.

16. Composition according to claim 1, characterized in that the cationic polymer(s) (v) are chosen from non-associative cationic polymers, and mixtures thereof, preferably from:
(1) homopolymers or copolymers derived from acrylic or methacrylic esters or amides and including at least one of the units having the following formulae: 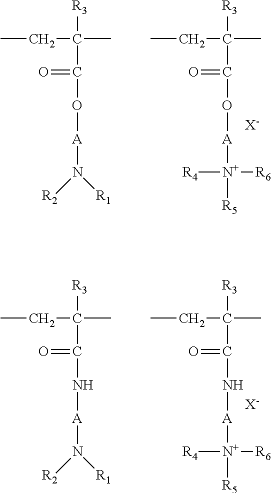
in which formulae:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical;
R₁ and R₂, which may be identical or different, represent a hydrogen atom or an alkyl group containing from 1 to 6 carbon atoms; and
X denotes an anion derived from a mineral or organic acid, such as a methosulfate anion or a halide such as chloride or bromide;
(2) cationic polysaccharides;
(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyamino amides prepared in particular by polycondensation of an acidic compound with a polyamine;
(5) polyamino amide derivatives resulting from the condensation of polyalkylene polyamines with polycarboxylic acids followed by alkylation with difunctional agents;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms;
(7) alkyldiallylamine or dialkyldiallylammonium cyclopolymers;
(8) diquaternary ammonium polymers comprising repeating units of formula (XI): 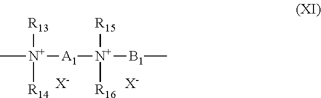
in which formula (XI):
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second non-nitrogen heteroatom, or alternatively R₁₃, R₁₄, R₁₅ and R₁₆ represent a linear or branched C₁ to C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ can also denote a group (CH₂)ₙCO-D-OC—(CH₂)ₙ— in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or alternatively the divalent radical —CH₂—CH₂—S—S—CH₂—CH₂—; or
d) a ureylene group of formula: —NH—CO—NH—;
(9) polyquaternary ammonium polymers comprising units of formula (XIII): 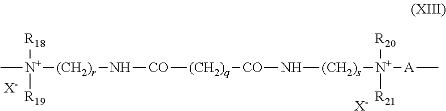
in which formula (XIII):
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion, such as a halide, and
A denotes a dihalide radical or preferably represents —CH₂—CH₂—O—CH₂—CH₂—;
(10) quaternary polymers of vinylpyrrolidone and of vinylimidazole;
(11) polyamines; and
(12) polymers including in their structure:
(a) one or more units corresponding to formula (A) below: 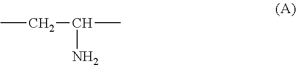
(b) optionally one or more units corresponding to formula (B) below: 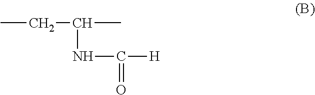
more preferentially chosen from cationic polysaccharides and mixtures thereof, even better still from cationic galactomannan gums and mixtures thereof, and even more preferentially from cationic guar gums and mixtures thereof.

17. Composition according to claim 1, characterized in that the total content of the cationic polymer(s) (v) ranges from 0.05% to 2% by weight, and preferably from 0.1% to 1% by weight, relative to the total weight of the composition.

18. Method for cosmetic treatment, in particular for washing and/or conditioning, of keratin fibres, notably human keratin fibres such as the hair, comprising the application to said keratin fibres of a composition as defined in claim 1; this application optionally being followed by rinsing after an optional leave-on time.

19. Use of a composition as defined in claim 1, for washing and/or conditioning keratin fibres, and in particular human keratin fibres such as the hair.
